(19) United States
(12) Patent Application Publication
(10) Pub. No.: US 2010/0215627 A1
(43) Pub. Date: Aug. 26, 2010

(54) ATOPY CURE BABY DRY MILK WITH GREEN TEA AND LACTOBACILLUS

(76) Inventors: Byung Yun Lim, Chungbuk (KR); Soo Hyun Lim, Chungbuk (KR); Yang Gyu Lim, Chungbuk (KR)

Correspondence Address: IPLA P.A., 3550 WILSHIRE BLVD., 17TH FLOOR, LOS ANGELES, CA, 90010, US

(21) Appl. No.: 12/680,802

(22) PCT Filed: Oct. 8, 2008
(86) PCT No.: PCT/KR08/05896

§ 371 Date: Mar. 30, 2010

(30) Foreign Application Priority Data
Oct. 10, 2007 (KR) 10-2007-0103324

(51) Int. Cl. A61K 35/74

(52) U.S. Cl. 424/93.45

(57) ABSTRACT

Provided is a prevention and treatment of baby atopy using green tea and lactobacillus digestives. By addition of 0.02˜1.3 wt % of green tea, and for the purpose of preventing side effects from green tea and increasing curative efficacy, addition of 0.15˜10 wt % (0.009˜0.8 wt % on pure lactobacillus basis) of lactobacillus digestives made up of lactobacillus or butyric-acid bacteria or yeast fungi to baby dry milk or weaning food, we can prevent and treat baby atopy. 0.1˜1.3 wt % of green tea content is adequate for the cure of babies' atopy and 0.02˜0.1 wt % of green tea content is adequate for the prevention and aftercare of baby atopy. Green tea or green tea extract and lactobacillus digestives are added during manufacturing process of the baby dry milk or weaning food.

### TECHNICAL FIELD

This is a new invention of baby dry milk and weaning food that cures and prevents baby atopy. There has been no medicine or food to cure atopy up to now. This invention is the addition of a certain portion of green tea and lactobacillus to baby dry milk or weaning food for the cure and prevention of atopy.

### BACKGROUND ART

Today atopy is known as one of the incurable diseases without a sure treatment, and many atopy patients are found among babies that have milk or weaning food.

Until now, atopy treatment has been largely limited to symptomatic treatment that temporarily controls eruption and inflammation appearing on skin surface by applying steroid drugs and moisturizer to patients' skin. But medicines or treatments that can fundamentally and easily cure atopy have not yet been invented for either babies or adults. A few oriental clinics practice treatment for atopy using their own herbal medicines, but their cure rate is very low. And they keep the composition secrete, require a long-term treatment of about 6 months. Some methods such as attaching green tea or aloes to patients' skin have different effects among every person and that is not established sure treatment.

To say nothing of medicines, there has been no baby dry milk or weaning food for the cure of baby atopy.

In particular, babies have no other choice but to eat one of mother's milk, baby dry milk, or infant food, so if they get atopy from these foods, they have to suffer a painful illness until they have grown up and have different types of food.

And green tea has caffein like coffee, so the general public and even doctors have had great aversion to giving green tea known as adult food to babies 1 year or under, the idea of mixing green tea with baby dry milk or weaning food could not be imagined, nor tested.

Having considered for a long time that the addition of green tea to baby dry milk or weaning food would have a good effect on babies, since the start of research several years ago, the inventor has been researching on the composition, applying for a patent. However, at the time of past application, the inventor hasn't thought about the effect of atopy cure, not to mention experimental approach. The inventor expected only the effect of general health improvement from green tea, and it was also impossible to apply the idea because it had not been tested.

And at the time of past application, it was not considered that lactobacillus digestives such as lactobacillus should be included to prevent side effects of green tea, so these recent research was not included in the last application.

The inventor's previous applications include one case related with lactobacillus, and two cases related with green tea.

Some baby food products containing lactobacillus are recently being released, but the inventor first applied for a patent on adding 0.0001-10 wt % and 0.5-10 wt % of lactobacillus to baby dry milk and weaning food for the purpose of improved intestinal digestion and prevention of harmful bacterium (Application No. 10-2004-0117551 “Baby dry milk and weaning food contain lactobacilli etc.”) on Dec. 28, 2004 when no one else had yet worked out the idea of adding lactobacillus to baby dry milk to help babies' intestinal digestion. It was much earlier than release of current few products contain lactobacillus. And it has not yet been confirmed whether content of lactobacillus featured in the current products was inspired by the inventor's previous application.

However, the inventor also admits that values of lactobacillus addition included in the previous application were at random without any reasonable basis, and had no idea that lactobacillus has additive effect of treating and preventing atopy as a supplement for green tea.

Any baby dry milk or weaning food with green tea contained has not yet been released up to now, but the inventor's previous applications include one on a product containing green tea as indicated below. The inventor has continued research related with green tea over several years and at last accomplished this invention of baby atopy treatment.

The inventor applied for a patent on “Green tea healthy food” (Application No. 10-2004-0097182) on Nov. 22, 2004. A part of the application includes description expecting the already well-known general effects of products with an arbitrary green tea content of 0.001-2.000 wt % in baby dry milk or weaning food. The inventor also made an application for “Green tea dry milk” (Application No. 10-2006-0069108) on Jul. 20, 2006. This was also one with an arbitrary green tea content of 0.01-0.50 wt % in baby dry milk or weaning food without any theoretical basis, only expecting the well-known general health improvement effects of green tea as in the Application No. 10-2004-0097182 above.

The purpose of adding green tea in baby dry milk or weaning food in the above two applications had nothing to do with the prevention and treatment of atopy this invention aims at,_and the green tea content (wt %) of milk products was arbitrary. And the above invention did not use lactobacillus for prevention of the side effects of green tea treatment, and it was impossible to apply to commercial manufacturing. Of course, the previous applications only featuring green tea contained never considered the special effect of curing infant atopy using green tea and lactobacillus included in the invention.

Based on the invention, when adding green tea and lactobacillus to baby dry milk or weaning foods, 0.02˜0.1 wt % of green tea is needed for products aimed at prevention of atopy, and 0.1˜1.3 wt % of green tea along with 0.15˜10 wt % (0.009˜0.8 wt % on pure lactobacillus basis) of lactobacillus digestive is needed for products aimed at cure baby atopy. These features of treating baby atopy using green tea in conjunction with lactobacillus is first invented in the application.

### DISCLOSURE

### [Technical Problem]

Existing baby dry milk and weaning food tend to cause allergy and atopy to babies due to its content of animal protein such as milk and eggs, or various artificial additives. However no food for atopy treatment has been invented up to now, and there is not any sure fundamental medical treatment, too. Steroid-kind of ointments given to patients in hospitals, unlike this invention, is only a makeshift that can't get rid of fundamental cause of atopy, but only remove visible symptoms such as eruption on the skin. So, many patients are seen to suffer from relapse just after some days of improvement, and what is worse is that long term use of those ointments can cause serious side effects.

Thus, this invention aims to provide babies with the baby dry milk and weaning food that can cure atopy and completely prevent atopy of normal babies who are given baby dry milk or weaning foods which can cause atopy easily, hoping to contribute to healthier society babies can be free from atopy.

### [Technical Solution]

To attain the purpose of treatment and prevention of baby atopy as in the above, this invention features mixing 0.02˜1.3 w/t wt % of green tea (on dry basis) in the product for the purpose of treatment and prevention of baby atopy. And when it comes to mixing green tea extract, green tea extract which is extracted from 0.02˜1.3 w/t wt % of green tea (on dry basis) should be mixed in products.

The adequate amount of green tea in baby dry milk or weaning food is 0.1-1.3 wt % in weight for treatment of baby atopy, and 0.02-0.1 wt % for prevention or aftercare of atopy.

For better curative effects from green tea and removal of a side effect of green stool caused by higher than 0.2 wt % of green tea concentration, 0.15-10 w/t wt % of lactobacillus digestives such as lactobacillus or butyric-acid bacteria, or yeast fungi (0.009-0.8 wt % on pure strain basis) would be added along. When lactobacillus digestives such as lactobacillus are added into baby dry milk or weaning food in conjunction with green tea, curative effects increase because of their co-supplementing function. Of course, it is recommended to increase the concentration of such lactobacillus digestives along when the concentration of green tea mixed is higher.

The applicant (inventor) himself opened an online cafe titled Atopy Baby Cure (http://cafe.naver.com/atopycure) for the prevention and cure of atopy in a local Internet search engine Naver on Mar. 6, 2007, and followingly was engaged in a long-term research of atopy treatment for over 6 months. Participants for the research test took part through various kinds of methods such as logging on the Internet Cafe, sending E-mails or on-line notes and telephone.

Through this research, the inventor found out that the use of lactobacillus digestive prevented side effects of green tea, and increased curative effects, and he posted this research results on the cafe starting Apr. 10, 2007.

The process of mixing green tea or green tea extract or lactobacillus digestives during manufacturing process of baby dry milk or weaning foods can be done as follows.

Manufacturing process of baby dry milk generally have follow steps. The first is raw material mixing followed by purification, sterilization, concentration, homogenization, drying, sieving, product mixing, nitrogen filling, and, at last, packaging. And each of adequate amount of green tea, green tea powder, liquid or solid form of green tea extract and lactobacillus digestives (strains with intestinal regulation and digestion functions such as lactobacillus and butyric-acid bacteria and yeast fungi) should be measured and added into the above process where it should be.

Atopy can be cured by adding only green tea without lactobacillus digestives, but if the content of green tea added is higher than 0.2 wt %, some useful intestinal bacterium would die or their intestinal activation can be impeded due to sterilizing function of green tea, which leads to green stool. Generally the higher concentration of green tea makes the thicker color of green stool. So it is much more recommendable to add lactobacillus digestives such as lactobacillus or butyric-acid bacteria, or yeast fungi to baby milk products along with green tea. If the content of green tea is lower than 0.2 wt %, you hardly have to worry about green stool, but since the addition of lactobacillus digestives increase the useful bacterium inside intestines, we can expect some reduction of negative effects caused by green tea.

The following is one of recommendable methods how to add in green tea and lactobacillus digestives such as lactobacillus during manufacturing process.

Green tea or green tea extract can be added into baby dry milk or weaning food in any process of the above entire process, but green tea powder or solid form of green tea extract are recommended to be added into during the drying or packaging process. And, if possible, it is recommended to set a mixing process of green tea and lactobacillus digestives by installing a mixing tank before or after sieving process. In that case, the green tea powder or dry green tea extract to be added in during the mixing process is recommended to be sterilized in advance. If green tea extract or lactobacillus digestives to be added into is solid-form, it is recommended to add them in granular form or powdered form.

Liquid green tea extract is recommended to be added in between the raw material mixing process and the drying process and higher concentration of green tea extract is recommended to be added in homogenization process after concentration, and lower concentration of green tea extract is recommended to be added in concentration process.

It is recommended that green tea leaves or green tea leaves crushed in a big size would be added in between the raw material mixing and the sterilization processes. At this time the most recommendable point of addition is right before the sterilization process. In that case of green tea leaves or leaves crushed in a big size used, the ingredients of green tea are extracted in hot milk during the processes so that another process is needed to remove big-sized leaves before the drying process. For that, it would be most recommendable to set a filtering process between the concentration process and the homogenization process. At this point, nitrogen purging can be considered to minimize the oxidization or browning of green tea extract during the manufacturing process.

Green tea powder can be added into in advance during the first half stage of the baby milk manufacturing process like the green tea leaves and leaves crushed in big size as shown in the above, and it can also be added into by setting a green tea mixing process before or after the sieving process in the second half stage of baby milk manufacturing process. If green tea powder is added into in the second half of the process like this, atopy is cured due to the effects of green tea itself, while if it is added into in the first half, green tea ingredients extracted into hot milk during manufacturing process remains in baby dry milk through concentration and drying. The choice of putting in green tea powder during the first half can require another process of filtering green tea powder, or let the green tea powder remain in baby dry milk to the last of manufacturing process. In this case, it is recommended to set another process of filtering green tea powder in the first half of the entire milk powder manufacturing process.

The general manufacturing process of baby weaning food is made up of mixing of raw materials such as baby dry milk, followed by drying, crushing, mixing, granulation, fraction, filling, and packaging. So, in this case it is recommended to add into green tea powder and dried lactobacillus in the mixing process. In the meantime, it is also possible to add dried green tea leaves during raw material crushing so that green tea leaves can be crushed with other raw materials.

The results of atopy treatment test using green tea extract and powder, based on this invention, was excellent as a whole with only a little difference case by case.

Green tea powder or fine pieces of green tea leaves are in general made using crushing or grinding device.

Crushing device is based on pressing or hitting mechanism with the pressing used for rough grinding and the hitting for both rough and fine grinding. Typical crushing devices are jaw crusher, hammer crusher, cutter, etc. Dried green tea leaves are easily broken with just a light pressing or hitting, which enables us to easily obtain small particles less than a few millimeters in diameter.

The most common grinding machines are ball mill, hammer mill, micro mill, and air jet mill. Green tea powder production can have grinding process after crushing process, or can only have grinding process.

These grinding machines can generally produce green tea powder particles of 1 to 100 μm in diameter with average diameter being 10 to 50 μm. This size of diameter of particles are adequate for applying the invention. In particular, if particles within specific range of diameter are required, you can go through a sifting screen for desired size after grinding and get more uniform size of green tea particles by recycling bigger than desired size of particles in the grinding process, and you get rid of smaller particles of green tea powder, and recycle them later when necessary, which allows you to adjust the size of particles as you want. In general, if particles are passed through 100-mesh screen with the thickness of mesh ignored, the diameter of passed particles becomes 254 μm with 25.4 mm divided by 100, but in reality only around 150 μm assuming the thickness of mesh is 100 μm.

Liquid or solid green tea extract can be obtained using solvents such as water and edible ethyl alcohol (ethanol) to extract green tea ingredients, which is used as it is, or concentrated, or dried after concentration. If water is used to extract green tea ingredients, the water is recommended to be first boiled to remove air and dissolved oxygen in it. If green tea is put in this hot water, you can reduce browning of the extract and extract most of useful ingredients in the green tea leaves.

Ethanol can be divided into edible and non-edible (for industrial use). Of course it is recommended to use edible ethanol when extracting green tea ingredients. Purified water, edible ethanol, or mixture of the two can be used as a solvent to extract green tea ingredients.

Below is a desirable method to extract green tea ingredients.

First, remove the oxygen dissolved in water by boiling a 1 m³ of purified water, from which residual cholorine has been in advance removed by passing through an activated carbon filter, at 1 atmospheric pressure and 100° C. or by applying vacuum heating. Second, put in the water 100-200 kg of dried green tea to extract green tea ingredients for 2-10 hours. Then, the concentration of this extract is 10-20 wt % by weight ratio. The higher the temperature of water, the more easily green tea ingredients are extracted. For more effective soak out, the desirable temperature of water would be higher than 60° C.

If the air and oxygen dissolved in the water are not removed, browning occurs easily. Of course, if vacuum heating is applied, the air and oxygen dissolved in the water can be removed even when water temperature does not reach 100° C.

The green tea extract produced in this manner can be used in a form of concentration, which requires atmospheric or vacuum distillation. At this point, fermented ethyl alcohol can be added to purified water for more effective soak out. Fermented ethyl alcohol is more expensive than water, but alcohol can be recycled after distilling the alcohol during distillation process. When adding ethyl alcohol, as the amount of the alcohol increases, green tea ingredients can be extracted more easily even at low temperature.

Since fermented alcohol is largely composed of ethanol (more than 95 wt %), but may contain a small amount of methanol, which is toxic substance harmful to health (* standard quality of fermented alcohol: methanol content of 0.5 mg/ml or less), it is recommended to extract green tea elements at a temperature lower than the boiling point of ethanol and higher than that of methanol if fermented alcohol is used.

As the boiling point of ethanol is 78.3° C. and that of methanol is 64.7° C., the temperature for the extraction of green tea ingredients recommended is between 65° C. and 75° C. at 1 atmospheric pressure. If water and alcohol are used combinedly as solvents for the extraction of green tea ingredients, it is recommended first to remove the air and dissolved oxygen in water before mixing.

In alcohol distillation process after extracting green tea ingredients, ethanol would be recovered through the primary distillation temperature which is 78.3-100° C. at 1 atmospheric pressure. Of course, depending on the atmospheric pressure set for a vacuum distillation process at 1 atmospheric pressure or less, ethanol can be first recovered, if distilled at higher temperature than the boiling point of ethanol and lower than that of water. Thus, cost can be saved by recycling the recovered ethanol.

After extracting green tea ingredients and removing alcohol in the primary distillation process, the extract is concentrated by heating at higher than the boiling point of water (it is 100° C. in case of 1 atmospheric pressure condition), if necessary.

Vacuum distillation enables energy saving due to easier distillation even at a lower temperature.

Solid form of green tea extract can be obtained by first concentrating the green tea extract obtained in the above, and secondly going through heating and drying process at atmospheric or vacuum pressure or a vacuum drying process, or a reduced pressure freeze-drying process, or a vacuum freeze-drying process.

Desirable methods for producing green tea extract have been introduced so far, but methods for extracting the green tea ingredients of this invention are not only limited to these methods.

Below are test examples that cured baby atopy using green tea.

### TEST EXAMPLE 1

A 7-month-old baby weighing 10 kg was cured of atopy after being fed with a 0.2-0.8 wt % concentration of green tea powder and extract for 3 weeks. The baby showed green stool after being fed for 2 days, but returned to normal stool after being fed with 2˜4 gram of lactobacillus digestives containing lactobacillus on daily basis. The baby started to be fed with yogurt purchasable on the market after about a month.

* Aftercare & Prevention—kept on aftercare using 0.15 wt % concentration of green tea for around a month from some 20 days after treatment, and good condition continued. The baby went into another aftercare stage using 0.04 wt % concentration of green tea powder and extract in around 2 months after treatment, and good condition continued.
* Special Features—this fat baby gained little weight during the treatment period.

### TEST EXAMPLE 2

A 6-month-old baby weighing 8.7 kg was completely cured after having 0.2-0.3 wt % concentration of green tea extract for 18 days. The baby was fed with 0.03 wt % concentration of green tea extract after treatment as aftercare and prevention measures.

### TEST EXAMPLE 3

A 4-month-old baby weighing 6.5 kg was completely cured after being fed with a 0.2 wt % concentration of green tea extract for a month, and showed yellow stool.

* Aftercare & Prevention: The baby was fed with a 0.02-0.13 wt % concentration of green tea extract, showing good health.

### TEST EXAMPLE 4

A 2-month-old baby weighing 5.2 kg was almost completely cured after having a 0.2 wt % concentration of green tea powder for a month.

* Treatment continued after the initial prescription: The baby showed good health after being fed with a 0.1 wt % of green tea powder for around 20 days, but not completely cured, either.
* Treatment continued after the second treatment: The baby was completely cured after receiving additional treatment with a 0.15 wt % concentration of green tea powder for around 20 days.
* Aftercare & Prevention: The baby was fed with a 0.05 wt % concentration of green tea powder for around 2 months, and a sign of no relapse

### TEST EXAMPLE 5

A 5-month-old baby weighing 6.9 kg was completely cured after receiving treatment with a 0.25 wt % concentration of green tea powder for around 5 weeks.

* Aftercare & Prevention: The baby was fed with a 0.02 wt % concentration of green tea extract.

### TEST EXAMPLE 6

A 5-month-old baby weighing 5.2 kg received treatment with a 0.1 wt % concentration of green tea powder for 2 weeks, which stopped without any sign of improvement seen.

* 0.1 wt % concentration of green tea powder was not sufficient for the babies in serious condition.

### TEST EXAMPLE 7

A 12-month-old baby weighing 10.5 kg was almost completely cured after receiving treatment with a 0.2 wt % concentration of green tea extract for a month.

* Finishing Treatment: The baby was completely cured after receiving additional treatment with a 0.15 wt % concentration of green tea extract for a month.

### TEST EXAMPLE 8

A 14-year-old teen improved significantly in health after receiving treatment with green tea leaves on the basis of 0.3 g/day for 2 weeks.

### TEST EXAMPLE 9

A 4-year-old child weighing 15 kg showed good condition after receiving treatment with a green tea extract on the basis of 0.4 g/day for 4 days.

### TEST EXAMPLE 10

A 5-month-old baby weighing 7 kg received treatment with a 0.15 wt % concentration of green tea extract for 10 days with clear symptomatic improvement not seen, but showed significant improvement after being fed with an increased green tea extract concentration of 0.3 wt % for 3 days, and showed good condition still after lowering to the 0.15 wt % concentration of green tea extract.

### TEST EXAMPLE 11

A 6-month-old baby weighing 8 kg showed no improvement with a 0.03 wt % concentration of green tea extract. The symptom improved after receiving treatment with 0.15 wt % concentration of green tea extract for around 2 weeks, but deteriorated when changing baby dry milk. This baby was cured after receiving treatment with an increased green tea extract concentration of 0.3 wt % for around 3 days.

* Aftercare & Prevention: Green tea powder with 0.01 wt % concentration was provided as an aftercare and prevention for around 2 months, but the concentration of 0.01 wt % seemed not sufficient for the baby concerned.

### TEST EXAMPLE 12

A 3-year-old child weighing 14.5 kg was almost completely cured after having green tea extract on the basis of 0.5 g/day for a week. After that, preventive aftercare was provided with the amount of green tea extract reduced to 0.3 g/day. The baby suffers from red atopy whenever he eats icecream, it feels so itchy that she has to scratch around.

### TEST EXAMPLE 13

A 6-year-old child showed improvement after receiving treatment with green tea extract on the basis of 0.3 g/day for 2 weeks.

### TEST EXAMPLE 14

A 5-year-old child showed improvement after receiving treatment with green tea extract on the basis of 0.3 g/day for 10 days.

### TEST EXAMPLE 15

A 5-month-old baby weighing 8 kg suffering from serious condition showed improvement after receiving treatment with a 0.4 wt % concentration of green tea powder, but that concentration seemed insufficient. The baby recovered to normal stool with lactobacillus digestives fed after the baby's green stool had occurred. A 0.6 wt % concentration of green tea powder brought remarkable improvement along with curative effects, but seemed insufficient.

0.8 wt % of concentration brought greater curative effects. The tester fed the baby with a 0.8 wt % concentration of green tea powder with good effects kept on, but once, by mistake, gave 0.6 wt % concentration of product unknowingly , which caused temporal deterioration. The baby's intake of green tea did not result in any improvement in atopy while the baby was suffering from a fever. The baby's health improved again when green tea powder with 1.0-1.3 wt % concentration was fed.

Since the baby's green stool kept on, the tester fed the baby with 2˜4 gram (1.5˜3 wt % of the baby dry milk) of lactobacillus digestives on daily basis, which helped return to normal stool. Since a sign of diarrhea was also found in that baby, the tester additionally fed the baby with fermented a lactobacillus digestive purchasable on the market.

The total amount of lactobacillus digestives fed reached 5 wt % or less of the baby dry milk on dry weight basis. The lactobacillus digestives used had in it 25 mg pure lactobacilli/g, 5 mg pure butyric acid bacteria/g, 50 mg pure dried yeast/g, and 3 mg pure activated digestive bacteria/g.

The above lactobacillus digestive had 8.3wt % of pure lactobacillus content rate so that 1.5˜3 wt % of the lactobacillus digestive is equal to 0.12˜0.25 wt % of lactobacillus on pure lactobacillus basis.

### TEST EXAMPLE 16

A 50-day-old baby weighing 5.9 kg received treatment with a 0.1-0.2 wt % concentration of green tea powder for around 20 days. The baby's health improved slightly even with a low green tea concentration of 0.1 wt %, and significantly improved with a green tea concentration of 0.2 wt % from the 3rd week of the treatment period.

The tests of atopy treatment were performed through various methods such as a cafe opened by the applicant in a Korean portal site Naver, E-mail, internet messenger, and telephone, and those who benefited from this invention include babies having mother's milk, baby dry milk, or baby weaning food as well as a few children.

Some babies suffering from other diseases such as Psoriasis which is not atopy also participated in the test as they misunderstood their symptom as atopy. But they did not show a sign of significant improvement. And atopy patients suffering from fevers such as flu also did not show significant curative effects. Except for these, all the other participants who complied with the applicant's treatment instruction were successfully cured within a month after treatment started.

The followed Table 1 and Table 2 are simplified descriptions of the test examples above.

TABLE 1
Summary of Treatment Period of Baby
Atopy and Green Tea Concentration

| | Green Tea | | |
|---|---|---|---|
| | Concentration | Treatment | |
| | (Weight wt %) | Period | Remarks |
| Test | 0.2~0.8 | 3 Weeks | Completely cured. |
| Example 1 | | | |
| Test | 0.2~0.3 | 3 Weeks | Completely cured. |
| Example 2 | | | |
| Test | 0.2 | 1 Month | Completely cured. |
| Example 3 | | | |
| Test | 0.2 | 1 Month | Completely cured. |
| Example 4 | | | |
| Test | 0.25 | 5 Weeks | Completely cured. |
| Example 5 | | | |
| Test | 0.1 | 2 Weeks | Treatment stopped (A 0.1 |
| Example 6 | | | wt % concentration of green |
| | | | tea was insufficient for |
| | | | babies in a serious condition.) |
| Test | 0.2 | 1 Month | Completely cured. |
| Example 7 | | | |
| Test | 0.15~0.3 | 2 Weeks | Significantly improved. |
| Example 10 | | | |
| Test | 0.15~0.3 | 3 Weeks | Completely cured. |
| Example 11 | | | |
| Test | 0.4~0.6 | 3 Weeks | Slightly improved, but green |
| Example 15 | | | tea concentration insufficient. |
| | 0.8~1.3 | 2 Weeks | Improved. |
| Test | 0.1~0.2 | 3 Weeks | Slightly improved with a |
| Example 16 | | | 0.1 wt % concentration of |
| | | | green tea for a 2-week period |
| | | | of treatment. Significantly |
| | | | improved with a 0.2 wt % |
| | | | concentration of green tea for |
| | | | a 1-week period of treatment. |

TABLE 2
Summary of Aftercare & Prevention Period after Baby
Atopy Treatment, and Green Tea Concentration

| | Green Tea | | | |
|---|---|---|---|---|
| | Concentration | | Treatment | |
| | (Weight wt %) | | Period | Remarks |
| Test | 0.04~0.15 | wt % | 3 Months | Showed good condition. |
| Example 1 | | | | |
| Test | 0.03 | wt % | 3 Months | Showed good condition. |
| Example 2 | | | | |
| Test | 0.02~0.13 | wt % | 3 Months | Showed good condition. |
| Example 3 | | | | |
| Test | 0.05~0.15 | wt % | 2 Months | Showed good condition. |
| Example 4 | | | | |
| Test | 0.02 | wt % | 2 Months | Showed good condition. |
| Example 5 | | | | |
| Test | 0.15 | wt % | 1 Month | Showed good condition. |
| Example 6 | | | | |
| Test | 0.01 | wt % | 2 Months | 0.01 wt % green tea |
| Example 11 | | | | concentration was weak and |
| | | | | slightly insufficient for |
| | | | | atopy treatment. |

So, to summarize the results of the above tests, baby dry milk or weaning food needs 0.1˜1.3 wt % of green tea concentration in order to treat atopy, and 0.02˜0.1 wt % of green tea concentration in order to prevent atopy. If you feed a baby with 0.15˜10 wt % (about 0.01˜0.8 wt % purely on lactobacillus basis) of lactobacillus digestives containing lactobacillus, butyric-acid bacteria or yeast fungi along with the baby dry milk or weaning food, you can prevent green stool which is a side effect of green tea and improve digestive function of a baby.

Valid lactobacilli are as follows; Reuteri, Lactobacillus, Bifidobacterium, Streptococcus, Leuconostoc, Pediococcus, Lactococcus, Lactobacillus Bulgaricus, Lactobacillus Casei, Lactobacillus acidophilus, Streptococcus Thermophilus.

Ingredients contained in one of the tested examples of baby dry milk are as follows. The each ingredient was measured by w/t percentage on dry basis.

0.02˜1.3 wt % of green tea or green tea extract, 1.5˜3.0 wt % of lactobacillus digestive containing lactobacillus or butyric-acid bacteria or yeast fungi, 40˜65 wt % of powdered whey, 1˜2 wt % of mixed edible fat, 10˜18 wt % of lactose, 20˜30 wt % of dry milk powder, 0.5˜1 wt % of galactosyl lactose, 0.5˜1 wt % of galacto-oligosaccharides, 0.3˜1.0 wt % of lactulose, 0.2˜0.5 wt % of casein, 0.28˜0.7 wt % of lecithin, 0.2˜0.5 wt % of L-cystine, 0.2˜0.5 wt % of L-arginine, 0.3˜0.5 wt % of vitamins and other nutrients.

Next is the ingredients of another tested example of baby dry milk.

0.02˜1.3 wt % of green tea or green tea extract, 1.5˜3.0 wt % of lactobacillus digestive containing lactobacillus or butyric-acid bacteria or yeast fungi, 40˜60 wt % of powdered whey, 1˜2wt % of vegetable fat, 10˜18 wt % of lactose, 25˜30 wt % of dry milk powder, 1.0˜2.0 wt % of galacto-oligosaccharides, 0.5˜1.5 wt % of egg yolk, 0.25˜0.7 wt % of lecithin, 0.23˜0.5 wt % of L-cystine, 0.2˜0.5 wt % of L-arginine, 0.3˜0.5 wt % of vitamins and other nutrients.

Ingredients contained in one of the tested examples of weaning food are as follows. The w/t percentage of each ingredient was measured on dry basis.

0.02˜1.3 wt % of green tea or green tea extract, 1.5˜3.0 wt % of digestive containing lactobacillus or butyric-acid bacteria or yeast fungi, 12˜15 wt % of organic brown rice, 5˜8 wt % of barley, 1˜2 wt % of millet, 1˜2 wt % of African millet, 1˜2 wt % of Proso millet, 2˜3 wt % of glutinous rice, 2˜3 wt % of parched beans, 20˜50 wt % of baby dry milk, 10˜15 wt % of whey protein, 2˜3 wt % of egg yolk, 1˜2 wt % of glucose, 0.6˜1 wt % of powdered apples, 0.7˜1 wt % of powdered tomatoes, 0.5˜1 wt % of powdered oranges, 0.6˜1 wt % of fructo-oligosaccharide, 0.3˜0.5 wt % of powdered seaweeds (such as brown seaweed, edible kelp), 0.3˜1.0 wt % of peas, 0.5˜1.0 wt % of chestnuts, 1.0˜1.5 wt % of beef, 0.5˜1.0 wt % of carrots, 0.3˜0.5 wt % of broccolis, 1.0˜1.5 wt % of squashs, 1.0˜1.5 wt % of mushroom, 1.0˜1.5 wt % of black forest mushroom, 0.5˜1.0 wt % of shrimps, 0.5˜1.0 wt % of galactosyl-lactose, 0.5˜1 wt % of galacto-oligosaccharide, 0.5˜1 wt % of lactulose, 0.2˜0.5 wt % of casein, 0.2˜0.5 wt % of lecithin, 0.2˜0.5 wt % of L-cystine, 0.2˜0.5 wt % of L-arginine, 0.02˜0.05 wt % of taurine, 0.02˜0.05 wt % of inositol, 0.02˜0.07 wt % of choline, 0.02˜0.03 wt % of gamma-linolenic acid, 0.3˜0.5 wt % of mixed vitamins (vitamin A B1 B2 B6 B12 C D3 E K1) and mixed nutrients.

The above describes the ingredients of tested examples used when green tea or green tea extract are used in conjunction with lactobacillus digestive containing lactobacillus or butyric-acid bacteria or yeast fungi. But it doesn't mean that the application is limited only to the range of the tested examples. So, when it comes to the proportion of ingredients to be mixed, various kinds of changeable range can be used as far as it doesn't hinder the essence of the invention. Therefore, it is clear that all such alteration should be subjected to the invention.

When you get to feed baby dry milk or weaning food with more than 0.2 wt % of dried green tea contained, you are recommended to feed lactobacillus digestive as well because it helps improve digestive function impeded by green tea and it can prevent green stool as well.

If the concentration of green tea in your baby dry milk or weaning food is lower than 0.2 wt % on dry weight basis, you don't have to worry about green stool in most cases. However it is more recommendable to mix lactobacillus digestive.

Lactobacillus, butyric-acid bacteria or yeast fungi help prevent green stool caused by green tea and help improve digestive function so that you can expect much better efficacy when those lactobacillus digestives are used in conjunction with green tea.

As a result of the experimental treatments, we could find out that green stool of babies was observed generally when the concentration of green tea was higher than 0.2 wt %. But when we fed the babies suffering from green stool with 2˜4 gram of lactobacillus digestive on daily basis (assuming that the average amount of daily consumed baby powder milk is 133 gram, the concentration of lactobacillus is equal to 1.5˜3.0 wt % of the baby powder milk.), we also found out that the babies were cured of green stool.

The concentration of green tea needed for prevention of atopy is at least 0.02 wt %, which is equal to one tenth of 0.2 wt %, the green tea concentration that causes green stool. So, applying the same proportion to get the adequate amount of the concentration of lactobacillus digestive needed, we need at least more than 0.15 wt % (0.01 wt % on pure lactobacillus basis) of lactobacillus digestive. And the upper limit for the concentration of green tea is 1.3 wt %, which is 6.5 times more than the 0.2 wt %. So applying the same proportion, we can know the upper limit for lactobacillus digestive concentration is 9.75 wt % (0.8 wt % on pure lactobacillus basis). That is why we decided the upper limit was 10 wt %.

The baby in the Test Example 1 above, who was fat, did not show any sign of weight increase during treatment while other babies showed weight increase. So it is expected that green tea helps prevent obesity.

Though other results we found out from the treatments was that, under 1.3 wt % content of green tea, almost all the babies who consistently took part in the treatments either were cured of atopy or showed improvement.

Except for a few babies who had other diseases, almost all the babies were completely cured of atopy or showed improvement about in a month.

It was found out that the least amount of green tea concentration needed for atopy treatment was 0.1˜1.3 wt % with the ideal amount varying among babies and the seriousness of atopy, and 0.02˜0.1 wt % of green tea concentration was adequate for prevention or aftercare of atopy.

The followed table is test data we got when we added different amount of lactobacillus digestive concentration into seven times diluted baby dry milk, which is almost equal to actual baby dry milk in concentration. It was done in a yoghurt_fermenter the temperature of which is 40 C, almost similar to body temperature. And the concentration of the lactobacillus digestive used in the experiment was 6 wt % on pure lactobacillus basis (containing 60 mg of lactobacilli every 1 g of digestive).

TABLE 3
Test for fermentation into yoghurt using lactobacillus

| Content of | | | | | |
|---|---|---|---|---|---|
| the | Concentration | observance time | | | |
| lactobacillus | of pure | after | after | after | |
| digestive | lactobacillui | 7 hours | 10 hours | 15 hours | etc |
| 0.1 | 0.006 | unfermented | begin | rotten | |
| | | | rotting | | |
| 0.15 | 0.009 | same as | not rotten | begin | |
| | | above | | rotting | |
| 0.2 | 0.012 | same as | same as | same as | |
| | | above | above | above | |
| 0.3 | 0.018 | same as | same as | same as | |
| | | above | above | above | |
| 0.5 | 0.03 | same as | same as | same as | |
| | | above | above | above | |
| 0.7 | 0.042 | same as | same as | same as | |
| | | above | above | above | |
| 1.0 | 0.06 | same as | same as | same as | |
| | | above | above | above | |
| 2.0 | 0.12 | same as | same as | same as | |
| | | above | above | above | |
| 3.0 | 0.18 | fermented | same as | same as | |
| | | into | above | above | |
| | | yoghurt | | | |
| 5.0 | 0.3 | same as | same as | same as | |
| | | above | above | above | |
| 7.0 | 0.42 | same as | same as | same as | |
| | | above | above | above | |
| 10.0 | 0.6 | same as | same as | same as | |
| | | above | above | above | |

As a result of the above experiment, it was found out that when the concentration of lactobacillus digestive was 0.1 wt % (0.006 wt % on pure lactobacillus basis), it started to rot 10 hours after the observance. So, it is thought that it has little effect on improving digestion. But it was found out that when the concentration of lactobacillus was higher than 0.15 wt % (0.009 wt % on pure lactobacillus basis, approximately 0.01 wt %), it started to rot as late as 15 hours after the observance. So it is thought that when the concentration is higher than 0.15 wt %, we can expect improvement of digestive function from it.

We can know from the above test that the concentration of lactobacillus digestive should be at least 0.15 wt % (0.009wt % on pure lactobacillus basis, approximately 0.01 wt %) to ensure improvement in digestive function.

This value exactly matches the 0.15 wt % of the least concentration needed for lactobacillus digestive we calculated in the above when we did test on babies. So we could confirm once more the least concentration of lactobacillus digestive needed to apply invention.

When the concentration of lactobacillus digestive was 3 wt % (0.18 wt % on pure lactobacillus basis), it was after 7 hours that liquid form was fermented into semi-solid form of yoghurt. But we can expect improved digestive function when the concentration of lactobacillus digestive is higher than 0.15 wt % (0.009wt % on pure lactobacillus basis).

Following the previous test, we made another test to confirm whether we could make yoghurt by adding 0.0˜1.3 wt % of green tea to baby dry milk when the concentration of lactobacillus digestive was 3 wt % (0.18 wt % on pure lactobacillus basis, approximately 0.2 wt %), which is the least concentration in which yoghurt could be made.

The test result is as follows.

TABLE 4
Test of adding green tea to baby dry milk with
3 wt % (0.18 wt % on pure lactobacillus basis)
concentration of lactobacillus digestive.

| The concentration of | Time taken to ferment | |
|---|---|---|
| green tea added (wt %) | into yoghurt (hours) | etc |
| 0.0 | 7 | |
| 0.1 | 10 | |
| 0.2 | 10 | |
| 0.3 | 10 | |
| 0.5 | 10 | |
| 0.7 | 10 | |
| 1.0 | 10 | |
| 1.3 | 10 | |

As you can see from the above result, baby dry milk without green tea added was fermented into yoghurt after 7 hours while baby dry milk with green tea added was fermented into yoghurt as late as after 10 hours. So we can know from the result that when we add green tea to lactobacillus digestive, we can expect digestive function certainly even if it is a little bit delayed by sterilizing effect of green tea. And through a beaker experiment, we could confirm the digestive function once more when we added lactobacillus to 1.3 wt % of green tea, which is the highest concentration of green tea available in the invention.

We did similar tests to this repeatedly. So when we added only 2 wt % of lactobacillus digestive to baby dry milk with the concentration of green tea being the same as in the previous experiment, we found out after 7 hours that semi-solid form of yoghurt was not made. And we added 5 wt % of lactobacillus digestive with the concentration of green tea being the same as in the previous experiment, we found out that semi-solid form of yoghurt was made after 7 hours.

For the purpose of comparison, we experimented on milk that lactobacillus digestive was added to it.

In the followed experiment (Table 5), when the concentration of lactobacillus digestive was 5wt %, some yoghurt was made even though as high as 1.3 wt % concentration of green tea was added. This is the decisive experimental evidence that digestive function occurs even if the content of green tea is as high as 1.3 wt %.

TABLE 5
Comparative experiment using milk

| | Concentration | Concentration | | |
|---|---|---|---|---|
| Content | of lactobacillus | of pure | Concentration | |
| of milk | digestives | lactobacillus | of green tea | |
| (ml) | (wt %) | (wt %) | (wt %) | result |
| 50 | 5 | 0.3 | 0.0 | fermented into |
| | | | | yoghurt after 8 hours |
| 50 | 5 | 0.3 | 0.1 | same as above |
| 50 | 5 | 0.3 | 0.2 | same as above |
| 50 | 5 | 0.3 | 0.3 | same as above |
| 50 | 5 | 0.3 | 0.5 | fermented into |
| | | | | yoghurt after 10 hours |
| 50 | 5 | 0.3 | 1.0 | only a little amount fermented |
| | | | | into yoghurt after 10 hours |
| 50 | 5 | 0.3 | 1.3 | same as above |
| 50 | 3 | 0.18 | 0.0 | fermented into |
| | | | | yoghurt after 8 hours |
| 50 | 3 | 0.18 | 0.1 | same as above |
| 50 | 3 | 0.18 | 0.2 | same as above |
| 50 | 3 | 0.18 | 0.3 | same as above |
| 50 | 2 | 0.12 | 0.0 | same as above |
| 50 | 1 | 0.06 | 0.0 | only a little amount fermented |
| | | | | into yoghurt after 8 hours |
| 50 | 0.5 | 0.03 | 0.0 | same as above |
| 50 | 0 | 0.00 | 0.0 | unfermented |

If addition of green tea deteriorated the taste of baby dry milk or weaning food then babies couldn't take it, so we tested for the change of taste as we added green tea into baby dry milk and weaning food each.

Test results shows next table 6

TABLE 6
Comparison of amount of green tea added to baby dry milk and the
change of taste

| | | | | Rate of taste | | | |
|---|---|---|---|---|---|---|---|
| | | | Concentration | (A: good, | | | |
| | Amount | Amount | of green | B: normal, | | | |
| Level | of green | of baby | tea | C: bad) | | | |
| of taste | tea added | dry milk | (wt %) | Gab | Eul | Byoung | Result |
| Level 1 | 0.0 g | 35 g | 0.0 wt % | A | A | A | sweet |
| | 0.2 g | 35 g | 0.6 wt % | A | A | A | a little bit |
| | | | | | | | unsweetened, |
| | | | | | | | but no taste |
| | | | | | | | of green tea |
| | 0.3 g | 36 g | 0.8 wt % | A | A | A | same as above |
| | 0.4 g | 40 g | 1.0 wt % | A | A | A | same as above |
| | 0.5 g | 40 g | 1.3 wt % | A | A | A | same as above |
| Level 2 | 0.6 g | 40 g | 1.5 wt % | B | B | B | significantly |
| | | | | | | | unsweetened, |
| | | | | | | | some taste of |
| | | | | | | | green tea |
| | 0.7 g | 40 g | 1.7 wt % | B | B | B | same as above |
| | 0.8 g | 40 g | 2.0 wt % | B | B | B | quite strong |
| | | | | | | | taste of |
| | | | | | | | green tea, |
| | | | | | | | but endurable |
| Level 3 | 0.8 g | 32 g | 2.5 wt % | C | C | C | sweetness |
| | | | | | | | hardly felt, |
| | | | | | | | strong touch |
| | | | | | | | of green tea |
| | | | | | | | tasted |
| | 0.9 g | 30 g | 3.0 wt % | C | C | C | very strong |
| | | | | | | | touch of |
| | | | | | | | green tea, no |
| | | | | | | | sweetness |

When we tested as in the above table, we could find out that the taste could be roughly divided into 3 levels. Up to 1.3 wt % of green tea concentration belongs to the first level of taste. In this level of taste, we could taste quite strong touch of sweetness and touch of green tea was hardly tasted so that babies had no problem with eating those, which is strongly supported by the case of a baby in the previous Test Example 15 who had no problem even eating baby dry milk with of as high as 1.3 wt % of green tea concentration.

It was already said that through various kinds of experiment, we got to receive 1.3 wt % as the upper limit of green tea concentration needed for treating babies' atopy. Coincidentally, that value matches the upper limit we got from this taste test.

When it comes to color, it was slightly yellow when green tea was not added, but as the concentration of green tea got higher, its color grew darker.

Measured on taste basis, the taste of baby dry milk with 1.5˜2.0 wt % of green tea concentration was deteriorated even though there was no serious problem with eating. But when the concentration of green tea was higher than 2.5 wt %, it was too hard to eat.

And the followed table 7 shows the change of taste of baby weaning food as the concentration of green tea changed.

TABLE 7
Comparison of amount of green tea added to baby weaning food and
change of taste

| | | | Concentration | Evaluation of taste | | | |
|---|---|---|---|---|---|---|---|
| Level | Amount of | Amount of | of | (A: good, B: normal, | | | |
| of | green tea | weaning | green | C: bad) | | | |
| taste | added | food\grave{ } | tea (wt %) | Gab | Eol | Byoung | Result |
| Level 1 | 0.0 g | 30 g | 0.0 wt % | A | A | A | strong taste |
| | | | | | | | of sweetness |
| | 0.18 g | 30 g | 0.6 wt % | A | A | A | a little |
| | | | | | | | unsweetened, |
| | | | | | | | no taste of |
| | | | | | | | green tea |
| | 0.2 g | 25 g | 0.8 wt % | A | A | A | same as above |
| | 0.3 g | 30 g | 1.0 wt % | A | A | A | a little |
| | | | | | | | unsweetened, |
| | | | | | | | slight taste |
| | | | | | | | of green tea |
| | 0.4 g | 30 g | 1.3 wt % | A | A | B | same as above |
| | 0.3 g | 20 g | 1.5 wt % | A | A | B | same as above |
| Level 2 | 0.5 g | 30 g | 1.7 wt % | B | C | C | clear taste |
| | | | | | | | of green tea, |
| | | | | | | | taste worse |
| | 0.6 g | 30 g | 2.0 wt % | B | B | C | same as above |
| | 0.75 g | 30 g | 2.5 wt % | B | C | C | slight |
| | | | | | | | sweetness, |
| | | | | | | | strong taste |
| | | | | | | | of green tea |
| Level 3 | 0.9 g | 30 g | 3.0 wt % | C | C | C | very strong |
| | | | | | | | taste of |
| | | | | | | | green tea |

When we experimented on the taste of weaning food as in the above table, we found out that the taste was largely divided into 3 levels. Up to 1.5 wt % of green tea concentration belongs to the first level of taste in which it feels quite sweet and touch of green tea is so weak that there is no problem with eating. But we are afraid that higher concentration of green tea is trouble to eat.

The value of 1.5 wt % is very close to the 1.3 wt % which is the upper limit for green tea concentration found in the tests done on babies.

When it comes to color, it was slightly yellow when green tea was not added. But as the concentration of green tea was higher, it grew darker.

Quality of green tea is rated according to how young its leaves are. So Ujeon made up of the first leaves picked before Kogu (about the 20th of April) is regarded as having the best quality. Followings are Sejak, Joongjak, Daejak in the order of quality.

And the quality is also influenced by what kind of method to use when growing green tea leaves. First method is a natural method to grow the leaves in the most similar environment to wild life, keeping as far from using agrichemicals as possible. Second is a Government-certified organic method not to use agrichemicals or synthesized fertilizers while using some natural fertilizers. Third is a general method to use both of agrichemicals and synthesized fertilizers.

When it comes to what kinds of green tea leaves to use for the invention, of course, it is recommendable to use such higher grades of leaves as Ujeon, Sejak, Joongjak. It is clear that we can expect quite high quality from Ujeon, but the price of Ujeon is very high. So it would be also wise to use Sejak or Joongjak at reasonable price. If you would like to use Daejak, which has low grade of quality, you should be aware that the property of lower grade of leaves could be a little bit strong rather than soft. So it is preferable not to use lower grade of leaves for the babies less than 50 days old. As an example of this, when we fed baby dry milk made up of Daejak, the lowest grade to a one-month-old baby, he had trouble eating it.

So, for babies less than 50 days old, it would be more recommendable to mix green tea extract rather than green tea itself. And for babies less than 100 days old, it would be more recommendable to lower concentration of green tea compared to those used for the babies more than 100 days old.

In this experiments, we used various kinds of green tea leaves grown from various environment from Ujeon leaves grown using the natural method to green tea powder made up of Sejak and Joongjak grown using the natural method, even to green tea powder made up of Joongjak and Daejak grown using the general method easily purchasable on the market. But there was no significant difference among efficacy of the treatment. For application to commercial manufacturing, of course, it would be most recommendable to use green tea grown using the natural method or the organic method.

We could find out that, for 6 months of test period, treatment of babies' atopy was quite successful when the concentration of green tea was within range of 0.1˜1.3 wt %. But treatment of atopy was impeded temporarily when a baby had a fever from other diseases.

When we mix lactobacillus digestives such as lactobacillus or butyric-acid bacteria or yeast fungi, we can expect better efficacy because those digestives prevent side effects caused by green tea. In addition, those useful lactobacilli help to destroy away various kinds of poisonous substances or poisonous bacterium. Breast-fed babies can expect improvement of digestive function because breast milk bears Reuteri, a kind of useful lactobacillus in it. But we can't expect this effect from baby dry milk or weaning food so that it is recommendable to add some useful lactobacillus for babies' health.

The causes and seriousness of atopy could vary among babies. So it is recommendable to diversify into more than 3 steps of products according to the concentration of green tea within range of 0.1˜1.3 wt % in case of products for atopycure. And for the purpose of prevention or aftercare of atopy, it is recommendable to diversify into more than 2 steps of products according to the concentration of green tea within range of 0.02˜0.1 wt % too.

### ADVANTAGEOUS EFFECTS

While many baby dry milk or weaning foods have the risk of causing atopy to babies, this invention enables complete prevention and cure treatment of babies' atopy which could not be done by even medical product.

When we tested for the invention, we also found out that children's atopy could be treated using the invention as well as babies' atopy.

Aside from the prevention and treatment of atopy, we can expect a lot of other advantageous effects from this invention. It includes: babies' emotional stability, improvement of babies' brain, prevention of children fatness, decline in blood fat and cholesterol, prevention of the proliferation of poisonous bacterium that could occur after opening of baby dry milk or weaning food, prevention of food poisoning and cancer, prevention of side effects caused by synthesized additive to baby dry milk or weaning food, prevention of decayed tooth, detoxication of various kinds of harmful substances that could be included for example cow's food originated substance, during manufacturing process.

In addition, when we mix lactobacillus digestives, babies can get improvement in digestive function that breast-fed babies get from Reuteri contained in breast milk. So, it is expected that the invention could contribute greatly to help the society healthier. As well, it could be used as a basis on which to start treatment atopy of children or adult.

### BEST MODE

Products aimed at treating atopy need to adjust concentration of green tea according to seriousness of atopy. Generally, 0.2 wt % of green tea concentration and 0.1 wt % (on pure lactobacillus basis) of lactobacillus digestive content is adequate for treating mild atopy. And 0.5 wt % of green tea concentration and 0.2 wt % (on pure lactobacillus basis) of lactobacillus digestive is adequate for treating common atopy. In the case of serious atopy, 0.8 wt % or green tea concentration with content of lactobacillus digestive being 0.3 wt % (on pure lactobacillus basis) is adequate.

For the products aimed at prevention or aftercare of atopy, 0.05 wt % of green tea concentration with content of lactobacillus digestive being 0.03 wt % (on pure lactobacillus basis) is adequate for prevention of atopy, and 0.1 wt % of green tea concentration with content of lactobacillus digestive being 0.05 wt % (on pure lactobacillus basis) is adequate for aftercare.

### MODE FOR INVENTION

When it comes to products aimed at treating atopy, it is recommendable to diversify the products into at least more than 3 steps according to concentration of green tea within range of 0.1˜1.3 wt %. And in the case of products aimed at prevention or aftercare of atopy, it is recommendable to diversify the products into at least more than 2 steps according to the concentration of green tea within range of 0.02˜0.1 wt %. For better treatment, it is much more recommendable to mix 0.15˜10 wt % (0.009˜0.8 wt % on pure lactobacillus basis) of lactobacillus digestives.

### INDUSTRIAL APPLICABILITY

Based on the invention, we experimented on babies using baby dry milk mixed with powdered green tea, which led to treatment of many babies' atopy. And we could also treat breast-fed babies' atopy using calculated amount of green tea extract. So, we can assure you that this invention can be applied to commercial manufacturing now.

1-6. (canceled)

7. For baby dry milk that characterize inclusion of green tea or green tea extract,
Baby dry milk that have 0.02˜1.3 weight % of green tea on a dry basis or green tea extract extracted from 0.02˜1.3 weight % of green tea on a dry basis added during the manufacturing process of the dry milk, in which case the total amount of green tea added directly and used for extraction is within range of a 0.02˜1.3 weight %; and
0.009˜0.8 weight % of lactobacilli, butyric acid or yeast fungi are mixed also during the manufacturing process of the baby dry milk.

8. For baby weaning food that characterize inclusion of green tea or green tea extract,
Baby weaning food that have 0.02˜1.3 weight % of green tea on a dry basis or green tea extract extracted from 0.02˜1.3 weight % of green tea on a dry basis added during the manufacturing process of the baby weaning food, in which case the total amount of green tea added directly and used for extraction is within range of a 0.02˜1.3 weight %; and
0.009˜0.8 weight % of lactobacilli, butyric acid or yeast fungi are mixed also during the manufacturing process of the baby weaning food.
